(19) United States
(12) Patent Application Publication
(10) Pub. No.: US 2025/0256124 A1
(43) Pub. Date: Aug. 14, 2025

(54) SYSTEM AND METHOD FOR HIGH PRESSURE DELIVERY OF RADIOACTIVE MATERIAL FOR CANCER THERAPY

(71) Applicant: BOSTON SCIENTIFIC SCIMED, INC., Maple Grove, MN (US)

(72) Inventors: Timothy A. OSTROOT, Cokato, MN (US); Summan RAHMAN, Murphy, TX (US); Shannon MCGILL, Friendswood, TX (US); Marin SCHIFFMAN, Lemont, IL (US); Aiden FLANAGAN, Kilcolgan (IE); Yuping WEI, Woodbury, MN (US)

(21) Appl. No.: 19/024,810

(22) Filed: Jan. 16, 2025

Related U.S. Application Data
(60) Provisional application No. 63/551,732, filed on Feb. 9, 2024.

(51) Int. Cl. A61N 5/10; A61B 34/20

(52) U.S. Cl. CPC A61N 5/1014; A61N 5/1048; A61B 2034/2063

(57) ABSTRACT

A system includes a device, a radioactive deliverable and a pressure source. The device includes a shaft and a needle received therein. The needle extends longitudinally from a proximal end to a distal end and is slidably received within a channel of the shaft. The needle is movable between an insertion configuration, in which a tip at the distal end thereof is housed within the channel of the shaft, and a piercing configuration, in which the needle is moved distally relative to the shaft so that the tip extends distally from the channel of the shaft to pierce a target tumor. The deliverable is inserted into a channel of the needle. The source is coupled to the proximal end of the needle to apply a pressure through the channel of the needle that is sufficient to inject the deliverable through the channel and into the tumor.

### PRIORITY CLAIM

The present disclosure claims priority to U.S. Provisional Patent Application Ser. No. 63/551,732 filed Feb. 9, 2024; the disclosure of which is incorporated herewith by reference.

### FIELD

The present disclosure relates to a system and method for cancer treatment and, in particular, relates to a system and method for endoscopic delivery of radioactive material for targeted cancer therapy.

### BACKGROUND

Non-small cell lung cancer accounts for approximately 85% of lung cancers and is the most common cause of cancer deaths. It arises from epithelial cells lining the airway and usually begins in the bronchi lining. Existing therapies may include, for example, surgery, resection, radiation therapy, chemotherapy, targeted drug therapy, and immunotherapy. In some cases, however, targeted treatment of non-small lung cancer may be difficult to achieve due to its location in deeper anatomies (e.g., smaller branches of the bronchial tree).

### SUMMARY

The present disclosure relates to a system for tumor ablation. The system includes a needle device comprising a shaft and a needle received therein, the shaft extending longitudinally from a proximal end to a distal end and including a channel extending therethrough, the needle extending longitudinally from a proximal end to a distal end and configured to be slidably received within the channel of the shaft, the needle movable between an insertion configuration, in which a tip at the distal end thereof is housed within the channel of the shaft, and a piercing configuration, in which the needle is moved distally relative to the shaft so that the tip extends distally from the channel of the shaft to pierce a target tumor.

The system also includes a radioactive deliverable configured to be inserted into a channel of the needle. In addition, the system includes a pressure source configured to be coupled to the proximal end of the needle to apply a pressure through the channel of the needle that is sufficient to inject the radioactive deliverable through the channel and into the target tumor.

In an embodiment, the shaft includes echogenic features along an exterior surface of a distal portion of the shaft.

In an embodiment, the shaft includes an ultrasound transducer extending distally from the distal end thereof.

In an embodiment, the shaft includes a needle indicator extending from the distal end of the shaft and visible via the ultrasound transducer to visualize a position of the needle relative to the shaft.

In an embodiment, the pressure is selected to achieve one of a desired depth of injection, dispersion, and leakage prevention of the radioactive deliverable.

In an embodiment, the radioactive deliverable is one of a microsphere embedded with a radioactive substance, a viscous gel including a radioactive substance, and a bioresorbable plug including a radioactive substance.

In an embodiment, the needle includes echogenic features along a portion of a length thereof.

In an embodiment, the tip of the needle includes one of a taper and a sharpened edge.

In addition, the present disclosure relates to a system for tumor ablation. The system includes a delivery device including a flexible shaft that is sized, shaped, and configured to be inserted through a bodily passageway to a target area within a patient. The system also includes a needle device configured to be inserted through a working channel of the delivery device to a target tumor in the target area, the needle device including a shaft and a needle slidably received therein, the shaft extending longitudinally from a proximal end to a distal end and including a channel extending therethrough, the needle extending longitudinally from a proximal end to a distal end and configured to be slidably received within the channel of the shaft, the needle movable between an insertion configuration, in which a tip at the distal end thereof is housed within the channel of the shaft, and a piercing configuration, in which the needle is moved distally relative to the shaft so that the tip extends distally from the channel to pierce the target tumor

In addition, the system includes a radioactive deliverable insertable through a channel of needle so that when a pressure is applied through the channel of the needle, the radioactive deliverable is injected into the target tumor.

In an embodiment, the delivery device is a bronchoscope.

In an embodiment, the shaft includes an ultrasound transducer extending distally from the distal end thereof.

In an embodiment, the pressure is selected to achieve one of a desired depth of injection, dispersion, and leakage prevention of the radioactive deliverable.

In an embodiment, the radioactive deliverable is one of a microsphere embedded with a radioactive substance, a viscous gel including a radioactive substance, and a bioresorbable plug including a radioactive substance.

In an embodiment, one of the shaft and the needle includes echogenic features along a portion of a length thereof.

In an embodiment, the tip of the needle is one of tapered and sharpened.

In addition, the present disclosure relates to a method for treating tissue. The method includes inserting a flexible shaft of a delivery device to a target area within a lung; inserting a needle device through a working channel of the delivery device to a target tumor in the target area; moving the needle device from an insertion configuration, in which a tip of a needle of the needle device is received within a channel of the shaft, toward a piercing configuration, in which the needle is moved distally relative to the shaft so that the tip of the needle pierces the target tumor; and injecting a radioactive deliverable received within the channel of the shaft into the target tumor by applying a pressure through a channel of the needle.

In an embodiment, inserting the needle device to the target tumor includes navigating the needle device toward the target tumor via ultrasound guidance.

In an embodiment, the method further includes positioning the needle device in a desired position relative to the target tumor via a visualization of a needle indicator which indicates a position of the needle relative to the shaft.

In an embodiment, the pressure is selected to achieve one of a desired depth of injection, dispersion, and leakage prevention of the radioactive deliverable.

In an embodiment, the radioactive deliverable is one of a microsphere embedded with a radioactive substance, a viscous gel including a radioactive substance, and a bioresorbable plug including a radioactive substance.

### BRIEF DESCRIPTION

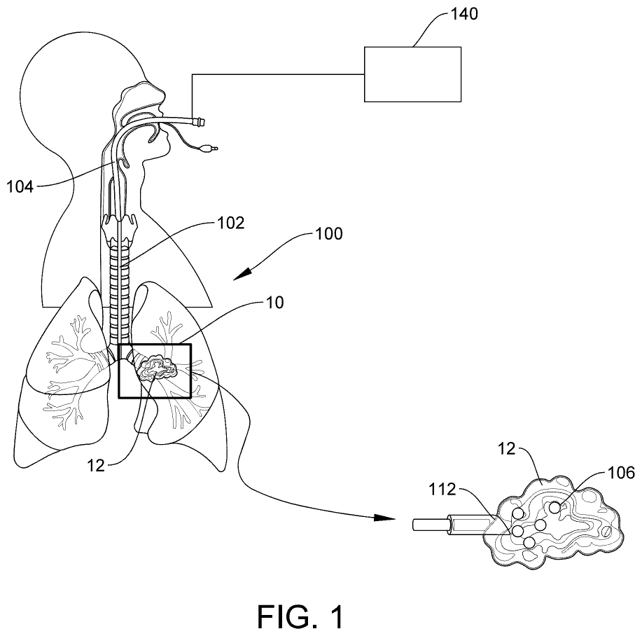

FIG. 1 shows a partially cross-sectional schematic side view of a system according to an exemplary embodiment of the present disclosure;

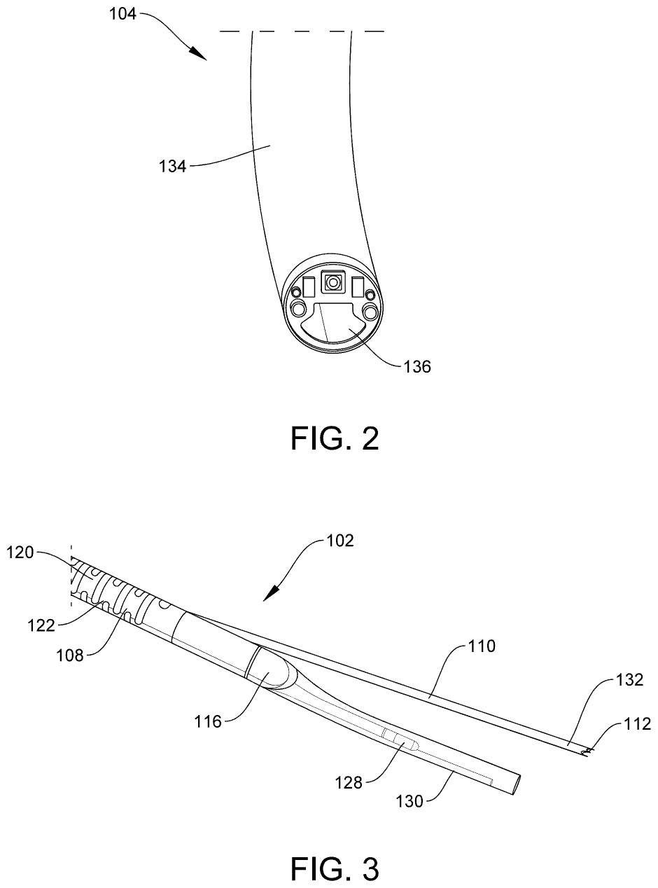

FIG. 2 shows a perspective view of a distal portion of a delivery device according to the exemplary system of FIG. 1;

FIG. 3 shows a side view of a distal portion of a needle device according to the exemplary system of FIG. 1;

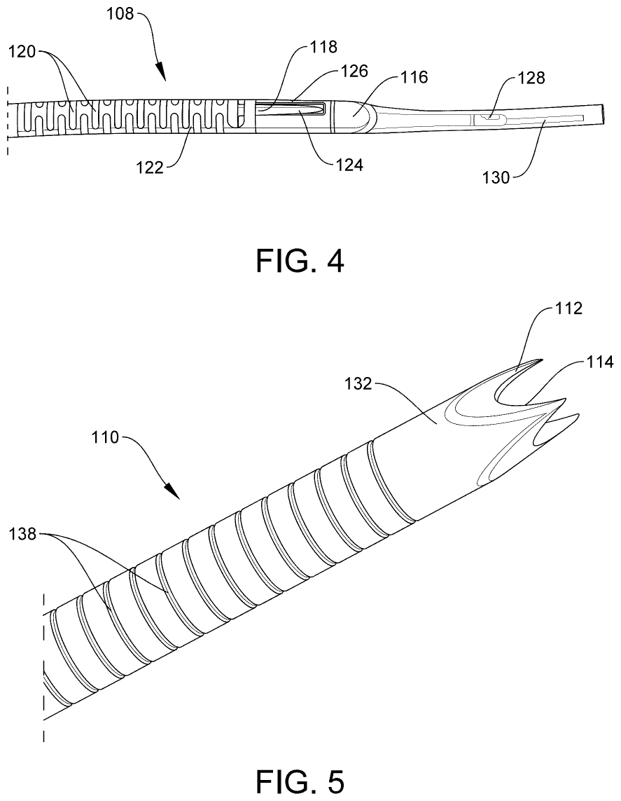

FIG. 4 shows a side view of a distal portion of a shaft of the needle device according to FIG. 3; and

FIG. 5 shows an enlarged side view of a distal portion of a needle of the needle device according to FIG. 3.

### DETAILED DESCRIPTION

The present disclosure may be further understood with reference to the following description and the appended drawings, wherein like elements are referred to with the same reference numerals. The present disclosure relates to a system and method for cancer treatment and, in particular, relates to a system and method for high pressure delivery of radioactive materials to a target area. Exemplary embodiments of the present disclosure comprise a needle device configured to be inserted to the target area to provide high pressure delivery of radioactive material into a tumor. The needle may be inserted to the target area via a delivery device such as, for example, a working channel of a scope device.

Although exemplary embodiments of the present disclosure are described as utilized for the treatment of non-small cell lung cancers, it will be understood by those of skill in the art that the systems and methods of the present disclosure may be similarly utilized for the treatment of various cancers that may benefit from targeted, high-pressure delivery of radioactive materials thereinto. It will also be understood by those of skill in the art that the terms proximal and distal, as used herein, are intended to refer to a direction toward (proximal) and away from (distal) a user of the device.

As shown in FIGS. 1-5, a system 100 for providing high pressure delivery of a radioactive deliverable 106 to target tissue (e.g., a target tumor 12) according to an exemplary embodiment comprises a needle device 102 configured to be inserted to a target area 10 via a delivery device 104 such as, for example, a bronchoscope or other scope device. Upon insertion of the delivery device 104 to the target area 10, the needle device 102 is inserted through a working channel of the delivery device 104 and guided to the target tumor 12 under, for example, ultrasound guidance. The needle device 102 includes a shaft 108 and a needle 110 slidable within the shaft 108.

The needle device 102 is configured to be moved distally out of the delivery device 104 and guided to the target tumor 12 via ultrasound guidance and/or guidance from a vision system of the delivery device. As shown in FIG. 1, the needle 110 includes a tip 112 configured to pierce the target tumor 12 so that the radioactive deliverable 106 is injectable through a channel 114 of the needle 110 into the target tumor 12, e.g., for tumor ablation. It will be understood by those of skill in the art that the radioactive deliverable 106 may provide targeted treatment of the target tumor 12 and/or may be used to provide pre-surgical intervention to reduce the size of the target tumor 12.

In an exemplary embodiment, the delivery device 104 is configured to be inserted to a target area 10 within a patient's body (e.g., within a lung) and may include, for example, a bronchoscope or other scope device (e.g., endoscope). As shown in FIG. 2, the delivery device 104 may include a flexible shaft 134 sized, shaped and configured to be passed through, for example, the nose or mouth, to the lung (e.g., via the trachea) and includes a visualization system including, for example, optical fibers, to provide visualization to a user (e.g., physician) of the airways of the patient. The flexible shaft 134 may be sized, shaped and sufficiently flexible to be insertable through bronchial passageways within the lung and includes a working channel 136 via which the needle device 102 may be inserted to the target area 10, as will be described in further detail below.

In an exemplary embodiment, the needle device 102 is configured to be inserted to the target area 10 via the working channel 136 of the delivery device 104. Upon arrival at the target area 10, the needle device 102 may be navigated to the target tumor 12 under ultrasound guidance as indicated above. The needle device 102 is sized, shaped, and configured to access distal bronchial passages and in one example, may have a 25 gauge. As described above and as shown in FIG. 3, the needle device 102 includes the shaft 108 and the needle 110.

As shown in FIGS. 3-4, the shaft 108 extends longitudinally from a proximal end (not shown) that remains outside the patient's body to a distal end 116 and includes a channel 118 extending therethrough. In an exemplary embodiment, a distal portion of the channel 118 includes a ramped surface 124 configured so that a distal opening 126 of the channel 118 extends through a wall of the shaft 108, proximally of the distal end 116 of the shaft 108. In other words, as the needle 110 is slid distally along the channel 118, the ramped surface 124 deflects the tip 112 of the needle 110 so that it moves out of the channel 118 with a portion of the needle 110 extending out of the shaft 108 angled with respect to a longitudinal axis of the shaft 108.

The ramped surface 124 of this embodiment facilitates insertion of the needle 110 into peripherally extending nodules/tumors (i.e., into tissue that is lateral the insertion device when the insertion device is inserted into a body lumen). In another exemplary embodiment, the channel 118 extends longitudinally through the shaft 108, from the proximal end to the distal end 116, so that the channel 118 is substantially aligned with the longitudinal axis of the shaft 108 and, so that the longitudinal axis of a portion of the shaft 108 within a body lumen is substantially parallel to the longitudinal axis of the body lumen at this location. Thus, the deflection of the tip 112 of the needle 110 allows the needle 110 to penetrate luminal tissue adjacent to a side wall of the shaft 108.

In an exemplary embodiment, the shaft 108 is formed of a metal material and is configured so that the shaft 108 is rotatable about the longitudinal axis thereof. In one example, the shaft 108 is formed of a braided metal material. The shaft 108 of this embodiment includes echogenic features 120 along an exterior surface of at least a distal portion 122 thereof to enhance a signal from an ultrasound imaging system to facilitate navigation of the needle device 102 to the target tumor 12. The echogenic features 120 may include, for example, surface features such as a coating, dimples, grooves, or bumps on an exterior surface of a distal portion 122 of the shaft 108.

In an exemplary embodiment, the needle device 102 further includes an ultrasound transducer 128 extending distally from the distal end 116 of the shaft 108. The ultrasound transducer 128 is configured to provide visualization as the shaft 108 is moved distally from the delivery device 104 toward the target tumor 12. In an exemplary embodiment, the ultrasound transducer 128 is a radial transducer configured to enhance visualization of peripheral tumors and/or nodules. In another exemplary embodiment, the ultrasound transducer 128 may include a forward-looking transducer.

The needle device 102 may further include an additional element such as, for example, a needle indicator 130 configured to facilitate a desired position of the needle device 102 relative to the target tumor 12 and/or to navigate the needle device 102 to the target tumor 12. According to an exemplary embodiment, the needle indicator 130 is configured to provide visualization of a relative position of the needle 110 relative to the shaft 108. The needle indicator 130 may be formed of a metal and may similarly extend distally from the distal end 116 of the shaft 108. In an exemplary embodiment, the needle indicator 130 may extend along the ultrasound transducer 128, along a side of a longitudinal axis of the shaft 108 opposite the distal opening 126 of the shaft 108 from which the needle 110 extends. It will be understood by those of skill in the art, however, that the needle indicator 130 may have any of a variety of configurations so long as the needle indicator 130 is visible via the ultrasound transducer 128 to indicate a position of the needle 110 and/or a position of the distal opening 126 of the channel 118 relative to the shaft 108. Thus, a user (e.g., physician) may visualize the position of the shaft 108 relative to the target tumor 12 to facilitate insertion of the needle 110 into the target tumor 12, as desired, and indicating an orientation of the shaft 108 so that, if necessary, the shaft 108 may be rotated to aim the needle 110 toward the target tissue.

According to another exemplary embodiment, the needle indicator 130 may be configured as a separate guidewire that is advanceable and/or torqueable relative to, for example, the shaft 108 of the needle device 102 to aid in distal nodule navigation and targeting. It will be understood by those of skill in the art, however, that the needle indicator 130 may have any of a variety of configurations so long as the needle indicator 130 aids in the positioning/navigation of the needle device 102 relative to the target tumor 12.

As shown in FIGS. 3 and 5, the needle 110 extends longitudinally from a proximal end (not shown) to a distal end 132 and includes the channel 114 extending therethrough from the proximal end to the distal end 132. The needle 110 is sized, shaped sufficiently flexible and otherwise configured to be slidable through the channel 118 of the shaft 108. In an insertion configuration, the needle 110 is housed within the shaft 108 such that the distal end 132 of the needle 110 is housed within the channel 118 of the shaft 108—i.e., the distal end 132 does not extend distally of the distal opening 126 of the channel 114 of the shaft 108. In a piercing configuration, the distal end 132 of the needle 110 is extended distally relative to the shaft 108 until the distal end 132 extends distally out of the distal opening 126 of the channel 118 to pierce the target tumor 12.

The distal end 132 includes the tip 112, which is configured to facilitate piercing of the target tumor 12. In an exemplary embodiment, the tip 112 may, for example, be tapered and/or include a sharpened edge. It will be understood by those of skill in the art that the tapering of the tip 112 may facilitate both a piercing of the target tumor 12 and, upon injection of the radioactive deliverable 106 into the target tumor 12, retention of the radioactive deliverable 106 within the target tumor 12. According to one example, the tip 112 may include a tube trocar. It will be understood by those of skill in the art, however, that the tip 112 may have any of a variety of configurations so long as the tip 112 is configured to puncture the target tumor 12, as described in further detail below.

The needle 110 of this embodiment is configured to extend from the distal end 116 of the shaft 108 and/or the distal opening 126 of the channel 118 of the shaft 108 via a desired length, corresponding, for example, to a desired depth of insertion of the tip 112 into the target tumor 12. It will be understood by those of skill in the art that this desired length may be adjusted based on a location, size and/or shape of the target tumor 12. In an exemplary embodiment, the needle 110 may similarly include echogenic features 138 along a portion thereof to increase visibility under ultrasound guidance. In one example, the echogenic features 138 may extend along a portion of the needle 110 which extends out of the shaft 108.

The needle 110 of this embodiment is formed of a metal material and is configured to shield surrounding portions of tissue (i.e., non-targeted tissue) from the radiation supplied by the radioactive deliverable 106 received in the needle 110 and passed therethrough to the target tumor 12. The metal material is also configured to withstand pressures required to deliver the radioactive material to the target tumor 12. According to an exemplary embodiment, the needle 110 may further include a metal lure attachment at the proximal end thereof, via which the radioactive deliverable 106 may be received within the channel 114 of the needle 110. As will be described in further detail below, the proximal end of the needle 110 is also configured to be connected to a pressure source 140 configured to provide pressurized delivery of the radioactive deliverable 106.

The radioactive deliverable 106 may be in any of a variety of configurations so long as the radioactive material is configured to provide targeted radiation and is suitable for insertion to the target tumor 12 via the channel 114 of the needle 110. In an exemplary embodiment, the radioactive deliverable 106 includes microspheres formed of, for example, glass or other suitable materials, which are embedded with a radioactive substance configured to provide targeted radioembolization. In another embodiment, the radioactive deliverable 106 includes a temporary and/or bioresorbable plug configured to be passed through the channel 114 of the needle 110. In yet another embodiment, the radioactive deliverable 106 is configured as a gel or fluid having a viscosity appropriate permitting it to be passed through the channel via high pressure delivery. It will be understood by those of skill in the art that the radioactive deliverable 106 is generally configured to provide a desired dosing tailored to the requirements of a specific patient.

In an exemplary embodiment, the radioactive deliverable 106 includes visualization markers configured to enhance tracking of the location at which the radioactive material has been injected into the tumor and/or to assess a dispersion of the radioactive deliverable 106 within the tumor. The visualization markers may include, for example, CT, Fluro fiduciaries, ultrasound additives, fluorescing markers, SPACE OAR gel type spacers. In another embodiment, the radioactive material has heating/cooling capabilities for controlling a viscosity of the radioactive deliverable 106 and a tissue take-up thereof.

As described above, the pressure source 140 is configured to be coupled to the needle 110 and may take any of a variety of configurations so long as the pressure source 140 is configured to provide pressure sufficient to inject the radioactive deliverable 106 through the needle 110 into the target tumor 12. In an exemplary embodiment, the pressure source 140 includes a pressure cartridge such as, for example, a multi-injection capable CO2 cartridge/syringe or a nitrogen cartridge. The pressure source 140 may be actuated via any of a number of actuators including, for example, an electronic pushbutton trigger.

As would be understood by those skilled in the art, the pressure source 140 is configured to provide a pressure sufficient to overcome a PSI drop due to, for example, a length and diameter of the needle 110 and/or a viscosity or form (e.g., solid) of the radioactive deliverable 106. As would also be understood by those skilled in the art, the delivery pressure is generally selected such that the radioactive deliverable 106 to be sufficient to inject the material to a desired depth within the target tumor 12 and/or to achieve a desired dispersion thereof. The delivery pressure may also be selected so as to prevent leakage of the radioactive material from the target tumor 12. In an exemplary embodiment, the pressure source 140 is shielded (e.g., formed of metal) to withstand any deleterious effects of the radioactive material of the radioactive deliverable 106. In an embodiment, the pressure source 140 is pre-irradiated and includes the radioactive deliverable 106.

According to a further exemplary embodiment, to prevent leakage of the radioactive deliverable 106 from the target tumor 12, the injection of the radioactive deliverable 106 into the target tumor 12 is followed up with, for example, a collagen plug, SpaceOAR, a viscous gel, or an expanding occlusion foam.

According to an exemplary method for providing targeted cancer treatment utilizing the system 100, the delivery device 104 (e.g., bronchoscope) is inserted into a target area 10 in a passageway of the lung via, for example, a nose or mouth of the patient. Upon reaching the target area 10, the needle device 102 may be guided to the target tumor 12, which is positioned further distally in the passageway, under ultrasound guidance. The shaft 108 of the needle device 102 according to an exemplary embodiment includes echogenic features 120 so that the needle device 102 is visible via ultrasound as it is moved toward the target tumor 12. As described above, the passageway and/or target tumor 12 may be visualized via the ultrasound transducer 128 at the distal end 116 of the shaft 108 as the needle device 102 is moved distally theretoward. The distal end 116 of the shaft 108 may be positioned adjacent and/or in contact with the target tumor 12.

Upon reaching the target tumor 12, the needle device 102 may be rotated, as necessary, so that the needle 110 is aimed toward the target tumor 12, as desired (i.e., so that a path of the needle 110 extends into the target tumor 12 when the tip 112 is moved distally out of the shaft 108). As described above, a relative position of the needle 110 relative to the shaft 108 may be visualized via the needle indicator 130. Once the shaft 108 has been positioned relative to the target tumor 12, as desired, the needle 110 is moved distally relative to the shaft 108 so that the tip 112 pierces the target tumor 12 and is inserted thereinto.

Upon insertion of the tip 112 into the target tumor 12, the radioactive deliverable 106 are inserted into the channel 114 of the needle 110 via, for example, a radioactive shielding luer attachment at the proximal end of the needle 110. Pressure may then be provided to the channel 114 via the pressure source 140 (e.g., pressure cartridge) connected to the proximal end of the needle 110 to inject the radioactive deliverable 106 into the target tumor 12. As described above, the pressure source 140 is generally configured to apply pressure sufficiently high to overcome PSI drops along the length of the channel 114 and/or caused via the radioactive deliverable 106 so that a desired depth of injection, dispersion and/or retention of the radioactive deliverable 106 in the target tumor 12 is achieved. In a further embodiment, upon injection of the radioactive deliverable 106, the target tumor 12 may be plugged via, for example, collagen, foam, viscous gel, etc., to prevent leakage of the radioactive deliverable therefrom.

Although the exemplary embodiments specifically show and describe treatment of non-small cell cancer and/or pulmonary cancer treatment, the systems and methods described herein may be utilized to treat alternate locations within the body and/or utilized with alternate modalities. For example, the needle device 102 may be inserted through a desired passageway without the use of an endoscope. Such an alternate system may include, for example, a guide catheter and/or a steering guide system. In another embodiment, the system 100 may include additional features for providing combination therapy. In one example, the delivery device 104 may include a wire attachment for impedance sensing for tumor detection, for providing RF application, and/or to provide a thermal IRE for tumor ablation. In an exemplary embodiment, the RF and/or IRE may be used for tumor “priming” prior to or following injection of the radioactive deliverable 106 to enhance the efficacy of the radioactive deliverable 106.

In addition, although the exemplary embodiments specifically describe the injection of radioactive deliverables 106, the system 100 may similarly be utilized for injection of a variety of different deliverable configurations. For example, deliverables may include pharmaceutical, drug, therapeutic, gene, virus, cell, protein, immunotherapy treatments.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A system for tumor ablation, comprising:
a needle device comprising a shaft and a needle received therein, the shaft extending longitudinally from a proximal end to a distal end and including a channel extending therethrough, the needle extending longitudinally from a proximal end to a distal end and configured to be slidably received within the channel of the shaft, the needle movable between an insertion configuration, in which a tip at the distal end thereof is housed within the channel of the shaft, and a piercing configuration, in which the needle is moved distally relative to the shaft so that the tip extends distally from the channel of the shaft to pierce a target tumor;
a radioactive deliverable configured to be inserted into a channel of the needle; and
a pressure source configured to be coupled to the proximal end of the needle to apply a pressure through the channel of the needle that is sufficient to inject the radioactive deliverable through the channel and into the target tumor.

17. The system of claim 16, wherein the shaft includes echogenic features along an exterior surface of a distal portion of the shaft.

18. The system of claim 16, wherein the shaft includes an ultrasound transducer extending distally from the distal end thereof.

19. The system of claim 18, wherein the shaft includes a needle indicator extending from the distal end of the shaft and visible via the ultrasound transducer to visualize a position of the needle relative to the shaft.

20. The system of claim 16, wherein the pressure is selected to achieve one of a desired depth of injection, dispersion, and leakage prevention of the radioactive deliverable.

21. The system of claim 16, wherein the radioactive deliverable is one of a microsphere embedded with a radioactive substance, a viscous gel including a radioactive substance, and a bioresorbable plug including a radioactive substance.

22. The system of claim 16, wherein the needle includes echogenic features along a portion of a length thereof.

23. The system of claim 16, wherein the tip of the needle includes one of a taper and a sharpened edge.

24. A system for tumor ablation, comprising:
a delivery device including a flexible shaft that is sized, shaped, and configured to be inserted through a bodily passageway to a target area within a patient;
a needle device configured to be inserted through a working channel of the delivery device to a target tumor in the target area, the needle device including a shaft and a needle slidably received therein, the shaft extending longitudinally from a proximal end to a distal end and including a channel extending therethrough, the needle extending longitudinally from a proximal end to a distal end and configured to be slidably received within the channel of the shaft, the needle movable between an insertion configuration, in which a tip at the distal end thereof is housed within the channel of the shaft, and a piercing configuration, in which the needle is moved distally relative to the shaft so that the tip extends distally from the channel to pierce the target tumor; and
a radioactive deliverable insertable through a channel of needle so that when a pressure is applied through the channel of the needle, the radioactive deliverable is injected into the target tumor.

25. The system of claim 24, wherein the delivery device is a bronchoscope.

26. The system of claim 24, wherein the shaft includes an ultrasound transducer extending distally from the distal end thereof.

27. The system of claim 24, wherein the pressure is selected to achieve one of a desired depth of injection, dispersion, and leakage prevention of the radioactive deliverable.

28. The system of claim 24, wherein the radioactive deliverable is one of a microsphere embedded with a radioactive substance, a viscous gel including a radioactive substance, and a bioresorbable plug including a radioactive substance.

29. The system of claim 24, wherein one of the shaft and the needle includes echogenic features along a portion of a length thereof.

30. The system of claim 24, wherein the tip of the needle is one of tapered and sharpened.

31. A method for treating tissue, comprising:
inserting a flexible shaft of a delivery device to a target area within a lung;
inserting a needle device through a working channel of the delivery device to a target tumor in the target area;
moving the needle device from an insertion configuration, in which a tip of a needle of the needle device is received within a channel of the shaft, toward a piercing configuration, in which the needle is moved distally relative to the shaft so that the tip of the needle pierces the target tumor; and
injecting a radioactive deliverable received within the channel of the shaft into the target tumor by applying a pressure through a channel of the needle.

32. The method of claim 31, wherein inserting the needle device to the target tumor includes navigating the needle device toward the target tumor via ultrasound guidance.

33. The method of claim 31, further comprising positioning the needle device in a desired position relative to the target tumor via a visualization of a needle indicator which indicates a position of the needle relative to the shaft.

34. The method of claim 31, wherein the pressure is selected to achieve one of a desired depth of injection, dispersion, and leakage prevention of the radioactive deliverable.

35. The method of claim 31, wherein the radioactive deliverable is one of a microsphere embedded with a radioactive substance, a viscous gel including a radioactive substance, and a bioresorbable plug including a radioactive substance.
